(19) United States
(12) Patent Application Publication
(10) Pub. No.: US 2014/0309470 A1
(43) Pub. Date: Oct. 16, 2014

(54) PREPARATION METHOD OF PLATINUM/TIN/ALUMINA CATALYST FOR DIRECT DEHYDROGENATION OF n-BUTANE AND METHOD FOR PRODUCING C4 OLEFINS USING SAID CATALYST

(71) Applicant: SAMSUNG TOTAL PETROCHEMICALS CO., LTD., Seosan-si (KR)

(72) Inventors: Gle PARK, Yongin-si (KR); Yeon Shick YOO, Seosan-si (KR); Young Jin CHO, Seosan-si (KR); Jin Suk LEE, Seoul (KR); Ho Sik CHANG, Daejeon (KR); Chang Hyun CHOI, Seoul (KR); In Kyu SONG, Seoul (KR); Jong Kwon LEE, Daejeon (KR); Hyun SEO, Daejeon (KR)

(73) Assignee: SAMSUNG TOTAL PETROCHEMICALS CO., LTD., Seosan-si (KR)

(21) Appl. No.: 14/249,955

(22) Filed: Apr. 10, 2014

(30) Foreign Application Priority Data
Apr. 11, 2013 (KR) 10-2013-0039749

(51) Int. Cl. B01J 23/62; C07C 5/32

(52) U.S. Cl. CPC B01J 23/626; C07C 5/325; C07C 2521/04; C07C 2523/42; C07C 2523/14
USPC 585/660; 502/334

(57) ABSTRACT

The provided is a preparation method of a platinum/tin/alumina catalyst which comprises platinum as an active component having high activity to direct dehydrogenation of n-butane, tin capable of preventing platinum particles from being sintered and maintaining a size of the platinum particles to be small, thereby improving dispersibility and increasing an amount at an active site during the dehydrogenation and also capable of suppressing carbon deposition, thereby increasing stability of the catalyst, and as an support for supporting them, an alumina support which is known as being suitable for direct dehydrogenation of n-butane and is capable of maintaining high dispersibility of the platinum with high thermal and mechanical stability, and a method for producing high value-added C4 olefins through direct dehydrogenation of inexpensive n-butane by using the catalyst prepared by the preparation method.

### CROSS-REFERENCE TO RELATED APPLICATION

This application claims the benefit and priority of Korean Patent Application No. 10-2013-0039749 filed Apr. 11, 2013. The entire disclosure of the above application is incorporated herein by reference.

### FIELD OF THE INVENTION

The present invention relates to a preparation method of a catalyst for direct dehydrogenation of n-butane, and more particularly, to a preparation method of a platinum/tin/alumina catalyst by impregnating tin and platinum in serial order on an alumina support, and a method for producing C4 olefins by using the catalyst.

### BACKGROUND OF THE INVENTION

In recent years, along with the rapid increase of demand for ethane-based crackers in the Middle East and the U.S.A., a structurally tight supply-demand balance for C4 olefins, particularly, butadiene has been worsened and a supply of C4 olefins has been limited. In particular, since the North America imported a shortfall in butadiene from Asia, butadiene was once sharply increased in price with a continued very strong trend. In addition to this, a limited supply of butadiene became an obstacle to extension of facilities for producing synthetic rubber, which resulted in imbalance between synthetic rubber supply and demand. At major sources of butadiene demand all over the world, demand increasing rate for a synthetic rubber is much higher than a butadiene supply increasing rate, and, thus, there is a concern over prolonged shortage of butadiene supply.

At present, most of C4 olefins including butadiene are produced by Naphtha Cracking Center (NCC) facilities all over the world by means of a steam cracking method. The steam cracking method is carried out at a high temperature of 800° C. or more and thus consumes a lot of energy accounting for about 40% of the total energy used in the petrochemical industry. Further, since the naphtha cracking center facilities are not only for producing C4 olefins, an expansion of production of C4 olefins cannot be expected by just newly and additionally installing naphtha cracking center facilities. Even if naphtha cracking center facilities are additionally installed, other basic fractions as well as C4 olefins are also produced. Therefore, a method for producing C4 olefins by the current naphtha cracking process is an energy-intensive process and cannot optimize a production process depending on a demand for C4 olefins, and, thus, it cannot be a fundamental method for producing C4 olefins.

In recent years, as the price of naphtha as a base material of producing C4 fractions has been increased, a base material of the method is changed to light hydrocarbon such as ethane, propane, etc. that has a low yield for production of C4 fractions but has a high yield for production of other basic fractions such as ethylene, propylene, etc., and a percentage of processes performed by the naphtha cracking center facilities has been relatively decreased. Therefore, it becomes more difficult to obtain C4 olefins through the naphtha cracking processes. Accordingly, there have been various needs for research on a method for producing C4 olefins without using naphtha cracking process. Dehydrogenation for obtaining C4 olefins by removing hydrogen from n-butane has drawn a lot of attention as a single process for producing C4 olefins which can be prompt in dealing with recent market changes, and currently, related researches have been conducted (Non-Patent Documents 1 to 4).

Dehydrogenation of n-butane is a reaction in which hydrogen is removed from n-butane to produce n-butene and 1,3-butadiene and can be classified into two types: direct dehydrogenation in which hydrogen is directly removed from n-butane; and oxidative dehydrogenation in which hydrogen is removed from n-butane by using oxygen. The oxidative dehydrogenation of n-butane is an exothermic reaction, and it has a thermodynamic advantage in that stable water is generated after the reaction. However, since the oxygen is used in the reaction, by-products such as carbon monoxide and carbon dioxide are generated during oxidation, and it is unfavorable in terms of selectivity and yield as compared with the direct dehydrogenation of n-butane. Meanwhile, the direct dehydrogenation of n-butane is an endothermic reaction, and it requires a higher reaction temperature than the oxidative dehydrogenation and uses a precious metal catalyst such as platinum. Since a catalyst has a very short life in many cases, the direct dehydrogenation needs a catalyst regeneration process. However, the direct dehydrogenation is known as a favorable process in terms of C4 olefin selectivity and yield (Patent Documents 1 to 4 and Non-Patent Documents 5 to 9).

Therefore, unlike the naphtha cracking process, the direct dehydrogenation of n-butane as a single process can produce C4 olefins, and if an appropriate catalytic process with high efficiency is developed, the direct dehydrogenation can be an effective alternative capable of reducing energy consumption and producing C4 olefins as a major product through a single process.

According to the above descriptions, the direct dehydrogenation of n-butane is favorable in terms of C4 olefin selectivity and yield as compared with the oxidative dehydrogenation, but it is expected that deactivation of a catalyst may occur due to coking and deposition as the reaction proceeds. Therefore, in order to commercialize the direct dehydrogenation of n-butane, it is important to develop a catalyst which can maintain a high conversion rate of n-butane with high selectivity and can also maintain a catalytic ability for a long time while suppressing deactivation due to coking and deposition.

Platinum/alumina-based catalysts (Patent Documents 1 to 4 and Non-Patent Documents 5 to 9), chrome/alumina-based catalysts (Patent Documents 5 to 6 and Non-Patent Document 9), vanadium-based catalysts (Non-Patent Documents 10 and 11), and the like have been used so far as catalyst systems for producing C4 olefins through the direct dehydrogenation of n-butane. The dehydrogenation of n-butane has been continuously researched since the late 1930s. In particular, during the Second World War, a process of producing C4 olefins from n-butane by using a chrome/alumina-based catalyst was developed in a process of production of octane for increasing an octane number of fuel, which set off the research on the direct dehydrogenation of n-butane. Since the 1960s, dehydrogenation of n-butane using a catalyst based on platinum as one of precious metals, for example, platinum/alumina-based catalyst has been continuously developed and researched, and since the 2000s, vanadium-based catalysts which can substitute for the expensive precious metal catalysts have been researched. It is known that among the above-described catalysts, the platinum/alumina-based catalysts have the highest activity in a reaction for direct dehydrogenation of n-butane and are the most suitable for the reaction (Non-Patent Document 9).

Typically, the platinum/alumina-based catalyst is produced in the form of platinum impregnated on alumina. To be specific, there has been reported a result of direct dehydrogenation of n-butane using 0.2 g of a platinum/alumina-based catalyst produced by impregnating platinum on commercial alumina (γ-Al₂O₃), and according to the report, dehydrogenation of n-butane was carried out at an injection ratio of hydrogen:n-butane of 1.25:1, at a total flow rate of 18 ml·min⁻¹, and at a reaction temperature of 530° C., and at the time of 10 minutes after the reaction, a n-butane conversion rate was 45%, C4 olefin selectivity was 53%, and yield was 24% and at the time of 2 hours after the reaction, a n-butane conversion rate was 10%, C4 olefin selectivity was 50%, and yield was 5% (Non-Patent Document 12). Further, it was reported that dehydrogenation of n-butane using 0.2 g of a platinum/alumina catalyst produced by using another alumina support (γ-Al₂O₃/α-Al₂O₃) was carried out at an injection ratio of hydrogen:n-butane of 1.25:1, at a total flow rate of 18 ml·min⁻¹, and at a reaction temperature of 530° C., and at the time of 10 minutes after the reaction, C4 olefin selectivity was 58.7% and yield was 18.3% and at the time of 2 hours after the reaction, C4 olefin selectivity was 62.4% and yield was 12.2% (Non-Patent Document 13).

If an enhancer is used in preparing a platinum/alumina catalyst, a catalytic activity can be improved by mutual combinations of platinum, an enhancer and an alumina support. As an agent for enhancing and stabilizing activity of platinum, tin has been usually used, and it was reported that a platinum/tin/alumina catalyst produced by impregnating platinum and tin on an alumina support has a good activity in direct dehydrogenation of n-butane. To be specific, there has been reported a result of direct dehydrogenation of n-butane using 0.2 g of a platinum/tin/alumina catalyst produced by impregnating platinum and tin in serial order on commercial alumina (γ-Al₂O₃), and according to the report, dehydrogenation of n-butane was carried out at an injection ratio of hydrogen:n-butane of 1.25:1, at a total flow rate of 18 ml·min⁻¹, and at a reaction temperature of 530° C., and at the time of 10 minutes after the reaction, a n-butane conversion rate was 43%, C4 olefin selectivity was 78%, and yield was 34% and at the time of 2 hours after the reaction, a n-butane conversion rate was 13%, C4 olefin selectivity was 86%, and yield was 11% (Non-Patent Document 12). Further, it was reported that dehydrogenation of n-butane using 0.2 g of a platinum/tin/alumina catalyst produced by using another alumina support (γ-Al₂O₃/α-Al₂O₃) was carried out at an injection ratio of hydrogen:n-butane of 1.25:1, at a total flow rate of 18 ml·min⁻¹, and at a reaction temperature of 530° C., and at the time of 10 minutes after the reaction, C4 olefin selectivity was 94.4% and yield was 28.5% and at the time of 2 hours after the reaction, C4 olefin selectivity was 95.9% and yield was 24.8% (Non-Patent Document 13). Furthermore, there has been reported a document in which sodium as another enhancer is used in a platinum/alumina catalyst (Non-Patent Document 14). According to this document, a sodium-added platinum/alumina catalyst was produced by adding sodium in an amount of 0.3 to 2 wt % to alumina and impregnating platinum, and 0.2 g of the produced catalyst was reduced by hydrogen at 530° C. for 3 hours and then applied to direct dehydrogenation of n-butane which was carried out at a hydrogen flow rate of 10 ml·min⁻¹, at a n-butane flow rate of 8 ml·min⁻¹, and at a reaction temperature of 530° C., and at the time of 10 minutes after the reaction, a n-butane conversion rate of 36.5% and C4 olefin selectivity of 74% was obtained from the platinum/alumina catalyst containing sodium in an amount of 0.3 wt % and at the time of 10 minutes after the reaction, a n-butane conversion rate of 8.2% and C4 olefin selectivity of 80% was obtained from the platinum/alumina catalyst containing sodium in an amount of 2 wt %.

There has been reported a document relating to a platinum/tin/alumina catalyst using method in which an additive is added to a platinum/tin/alumina catalyst produced by impregnating tin and platinum on alumina to improve activity in direct dehydrogenation of n-butane, and, thus, C4 olefins can be obtained with high selectivity and high yield (Non-Patent Document 15). According to this document, a sodium-added platinum/tin/alumina catalyst was produced by adding sodium to commercial alumina and impregnating platinum and tin, and 0.2 g of the produced catalyst was reduced by hydrogen at 530° C. for 3 hours and then applied to a reaction for direct dehydrogenation of n-butane which was carried out at a total flow rate of 18 ml·min⁻¹, at an injection ratio of hydrogen:n-butane of 1.25:1, and at the time of 10 minutes after the reaction of the platinum/tin/alumina catalyst containing sodium in an amount of 0.3 wt %, a n-butane conversion rate of 34% and C4 olefin selectivity of 96% was obtained and at the time of 2 hours after the reaction, a n-butane conversion rate of 19% and C4 olefin selectivity of 97% was obtained.

If the platinum/tin/alumina catalyst containing platinum and tin impregnated on alumina is used in carrying out direct dehydrogenation of n-butane, C4 olefins can be obtained with high selectivity and high yield, but during the catalytic reaction, deactivation may occur due to coking and deposition, and high activity of the catalyst cannot be maintained for a long time. Therefore, a catalyst capable of maintaining catalytic performance for a long time needs to be developed.

Hence, the present inventors established a preparation method of a platinum/tin/alumina catalyst containing platinum and tin impregnated on alumina based on their research. The preparation method can minimize a conventional problem of a decrease in activity of a catalyst with time and can prepare the catalyst through an inexpensive and simple process. If the prepared catalyst is applied to direct dehydrogenation of n-butane, the catalyst has high activity with respect to C4 olefins and particularly during the reaction, deactivation of the catalyst less occurs. By using the catalyst prepared as such, a catalytic reaction process capable of suppressing deactivation and producing C4 olefins with high selectivity and high yield was developed. Further, by establishing the preparation method of a platinum/tin/alumina catalyst through a simple process, reproducibility in preparing the catalyst can be attained.

### SUMMARY OF THE INVENTION

An object of the present invention is to provide a simpler and reproducible preparation method of a platinum/tin/alumina catalyst by impregnating tin and platinum on commercial alumina in serial order, and when the platinum/tin/alumina catalyst is applied to direct dehydrogenation of n-butane, deactivation of the platinum as an active component less occurs and high activity can be obtained.

Another object of the present invention is to provide a method for producing C4 olefins through carrying out direct dehydrogenation of n-butane with high activity and suppressed deactivation by using the platinum/tin/alumina catalyst prepared by the preparation method.

### DETAILED DESCRIPTION OF THE INVENTION

In order to achieve the above object, the present invention provides a preparation method of a platinum/tin/alumina catalyst for direct dehydrogenation of n-butane comprising the following steps:

(a) preparing a tin precursor solution by dissolving a tin precursor and an acid in a first solvent;

(b) impregnating the tin precursor solution on a basic alumina support;

(c) obtaining tin/alumina in which tin is impregnated on the alumina support by heat drying and heat treating a product obtained from the step (b);

(d) preparing a platinum precursor solution by dissolving a platinum precursor in a second solvent;

(e) impregnating the platinum precursor solution on the tin/alumina prepared in the step (c); and

(f) obtaining the platinum/tin/alumina catalyst for direct dehydrogenation of n-butane by heat drying and heat treating a product obtained from the step (e).

The tin precursor used in the step (a) may be any precursor as long as it is a precursor typically used. In general, it is desirable to use at least one selected from a group consisting of tin chloride, tin nitride, tin bromide, tin oxide and tin acetate. In particular, it is desirable to use the tin chloride (tin(II) chloride).

An amount of the tin precursor used in the step (a) is not particularly limited, but in order to prepare the platinum/tin/alumina catalyst capable of maintaining high activity for a long time as the object of the present invention, a tin content is 0.5 to 10 wt %, and desirably 1 wt %, based on the total weight of a final platinum/tin/alumina catalyst. If tin is added in an amount of more than 10 wt %, platinum active sites in the prepared catalyst are decreased, thereby resulting in a decrease in catalytic activity, which is not desirable. Meanwhile, if tin is added in an amount of less than 0.5 wt %, the tin does not perform its ability for preventing the platinum from being sintered and maintaining a small particle size of the platinum, thereby resulting in improvement of dispersibility and suppressing carbon deposition, which is not desirable.

The acid used in the step (a) exists at room temperature in the form of a liquid (solution) and may be, but not limited to, at least one selected from a group consisting of hydrochloric acid, nitric acid, sulfuric acid, hydrofluoric acid and phosphoric acid.

The first solvent and the second solvent respectively used in the step (a) and the step (d) may be selected from, but not limited to, water and alcohol, and desirably may be water.

Preferably, the alumina used in the step (b) may be basic alumina. Typically, alumina can be classified into acidic alumina, neutral alumina, and basic alumina. Herein, the criteria for classification to acidic alumina, neutral alumina and basic alumina may be a pH in a state where alumina is dispersed in water, and the basic alumina has a pH of 9.0 to 10.0, the neutral alumina has a pH of 6.5 to 7.5, and the acidic alumina has a pH of 6.0 or less. Among the acidic alumina, alumina having a pH of 4.0 to 6.0 may be classified as weakly acidic alumina.

The present invention first discloses that since the basic alumina is used, a catalyst having high activity and high C4 olefin selectivity can be prepared.

The purpose of the heat drying in the step (c) is to remove remaining moisture after impregnating the tin. Therefore, a drying temperature and a drying time can be determined according to typical drying conditions for removing moisture. For example, a drying temperature may be determined in a range of 50 to 200° C., and preferably 70 to 120° C., and a drying time may be determined in a range of 3 to 24 hours, and preferably 6 to 12 hours.

Further, the purpose of the heat treatment in the step (c) is to synthesize tin/alumina, and the heat treatment is carried out at 350 to 1000° C., and preferably at 500 to 800° C. for 1 to 12 hours, and preferably for 3 to 6 hours. If the heat treatment is carried out at a temperature lower than 350° C. or for a time shorter than 1 hour, tin/alumina is not sufficiently synthesized, which is not desirable. If the heat treatment is carried out at a temperature higher than 1000° C. or for a time longer than 12 hours, a phase of tin/alumina can be modified, which is not desirable.

The platinum precursor used in the step (d) may be any precursor as long as it is a precursor typically used. In general, it is desirable to use at least one selected from a group consisting of chloroplatinic acid, platinum oxide, platinum chloride and platinum bromide. In particular, it is desirable to use the chloroplatinic acid.

Further, an amount of the platinum precursor used in the step (d) is not particularly limited, but in order to prepare the platinum/tin/alumina catalyst capable of maintaining high activity for a long time as the object of the present invention, a platinum content is 0.5 to 10 wt %, and preferably 1 wt %, based on the total weight of the final platinum/tin/alumina catalyst. If platinum is added in an amount of more than 10 wt %, it becomes difficult to obtain a catalyst having high dispersibility of the platinum and expensive platinum may be overused, which is not desirable. Meanwhile, if platinum is added in an amount of less than 0.5 wt %, active sites of the platinum as an active metal for the direct dehydrogenation of n-butane are not sufficiently formed and it becomes difficult to produce C4 olefins with high selectivity and high yield, which is not desirable.

The purpose of the heat drying in the step (f) is to remove remaining moisture after impregnating the platinum. Therefore, a drying temperature and a drying time can be determined according to typical drying conditions for removing moisture. For example, a drying temperature may be determined in a range of 50 to 200° C., and preferably 70 to 120° C., and a drying time may be determined in a range of 3 to 24 hours, and preferably 6 to 12 hours.

Further, the heat treatment in the step (f) can be carried out at a temperature of 400 to 800° C. for 1 to 12 hours, and preferably at a temperature of 500 to 700° C. for 3 to 6 hours, whereby the platinum/tin/alumina catalyst can be obtained. The purpose of the heat treating the dried solid sample is not only to obtain platinum/tin/alumina catalyst but also to suppress modification of the catalyst when the prepared catalyst is used in the direct dehydrogenation of n-butane considering a reaction temperature of the reaction. If the heat treatment is carried out at a temperature lower than 400° C. or for a time shorter than 1 hour, the platinum/tin/alumina catalyst is not sufficiently formed, which is not desirable. If the heat treatment is carried out at a temperature higher than 800° C. or for a time longer than 12 hours, a crystalline phase of the platinum/tin/alumina catalyst can be modified to a phase unsuitable to be used as a catalyst, which is not desirable.

Further, exemplary embodiment of the present invention provides a method for producing C4 olefins through direct dehydrogenation of n-butane on a platinum/tin/alumina catalyst prepared by the above-described method.

A reactant of the direct dehydrogenation of n-butane is a mixed gas containing n-butane and nitrogen, and a volume ratio of the n-butane to the nitrogen is 1:0.2 to 10, and preferably 1:0.5 to 5, and more preferably 1:1. If a volume ratio of the n-butane to the nitrogen is out of the above-described range, deactivation may rapidly occur due to coking during the direct dehydrogenation of n-butane, or activity or selectivity of the catalyst may be decreased, resulting in a decrease in production of C4 olefins, and there may be a problem with safety of the process, which is not desirable.

When the reactant in the form of a mixed gas is supplied to a reactor, an injection amount of the reactant can be regulated by using a mass flow controller. The injection amount of the reactant may be set such that a Weight Hourly Space Velocity (WHSV) can be 10 to 6000 cc·hr⁻¹·gcat⁻¹, and preferably 100 to 3000 cc·hr⁻¹·gcat⁻¹, and more preferably 300 to 1000 cc·hr⁻¹·gcat⁻¹ with respect to the n-butane. If a weight hourly space velocity is less than 10 cc·hr⁻¹·gcat⁻¹, production of C4 olefins is too small, which is not desirable. If a weight hourly space velocity is more than 6000 cc·hr⁻¹·gcat⁻¹, coking and deposition rapidly occurs due to by-products of the reaction, which is not desirable.

A reaction temperature for carrying out the direct dehydrogenation of n-butane may be desirably 300 to 800° C. and more preferably 500 to 600° C., and it is most desirable to maintain a reaction temperature at 550° C. If the reaction temperature is lower than 300° C., a reaction of the n-butane is not sufficiently activated, which is not desirable. If the reaction temperature is higher than 800° C., a decomposition reaction of the n-butane mainly occurs, which is not desirable.

According to the present invention, a platinum/tin/alumina catalyst can be easily prepared through a simple preparation method, and a high reproducibility in preparing the catalyst can be attained.

Further, by using the platinum/tin/alumina catalyst according to the present invention, it is possible to produce C4 olefins, which has been gradually increased in demand and value all over the world, from n-butane which is less useful. Thus, it is possible to add a high value to C4 fractions.

Furthermore, since a single production process capable of producing C4 olefins by using the platinum/tin/alumina catalyst according to the present invention is obtained, an increasing demand for C4 olefins can be met without newly installing naphtha cracking center facilities, whereby financial benefits can be gained.

### BRIEF DESCRIPTION OF THE DRAWINGS

FIG. 1 is a graph showing a difference in yield among platinum/tin/alumina catalysts during direct dehydrogenation of n-butane carried out on each of the platinum/tin/alumina catalysts according to Example 1 and Comparative Example 1 for 270 minutes;

FIG. 2 is a graph showing a difference in selectivity among platinum/tin/alumina catalysts during direct dehydrogenation of n-butane carried out on each of the platinum/tin/alumina catalysts according to Example 1 and Comparative Example 1 for 270 minutes; and

FIG. 3 is a graph showing a distribution chart of products with respect to reactants of direct dehydrogenation of n-butane using respective platinum/tin/alumina catalysts after the direct dehydrogenation of n-butane carried out on each of the platinum/tin/alumina catalysts according to Example 1 and Comparative Example 1 for 270 minutes.

### EMBODIMENTS FOR PERFORMING THE INVENTION

Hereinafter, the present invention will be explained in more detail with reference to exemplary embodiments. However, these exemplary embodiments are provided just for illustration, but do not limit the present invention.

### Preparation Example 1

### Preparation of Platinum/Tin/Alumina (Pt/Sn/Al₂O₃(B)) Catalyst by Impregnating Tin and Platinum in Serial Order on Basic Alumina (Aluminum Oxide)

By using basic alumina (aluminum oxide; manufactured by ACROS) to prepare tin/alumina impregnating tin to be in an amount of 1 wt %, 0.038 g of tin(II) chloride dihydrate as a tin precursor was put into a beaker to be dissolved in a small amount of hydrochloric acid (0.37 ml) and distilled water (15 ml). When the tin precursor was completely dissolved in the solution prepared as such, 2.0 g of the basic alumina was added thereto and heated at 70° C. with stirring until the distilled water was completely evaporated. As a result, a solid material remained. Then, the solid material was additionally dried in an oven at 80° C. for 12 hours, and a resultant sample was heat treated in an electric furnace in an air atmosphere at 600° C. for 4 hours. As a result, tin/alumina impregnating tin in an amount of 1 wt % on alumina was obtained.

0.053 g of chloroplatinic acid hexahydrate as a platinum precursor was put into a beaker to be dissolved in distilled water (10 ml) such that a platinum content could be 1 wt % in 2.0 g of the tin/alumina sample obtained as such. When the platinum precursor was completely dissolved in the solution, 2.0 g of the prepared tin/alumina was added to the precursor solution and then stirred at 70° C. until the distilled water was completely evaporated. Then, a remaining solid material was additionally dried in an oven at 80° C. for 12 hours, and a resultant sample was heat treated in an electric furnace in an air atmosphere at 550° C. for 4 hours. As a result, a platinum/tin/alumina catalyst was prepared, and the prepared catalyst was named Pt/Sn/Al₂O₃(B).

### Comparative Preparation Example 1

### Preparation of Platinum/Tin/Alumina (Pt/Sn/Al₂O₃(N)) Catalyst by Impregnating Tin and Platinum in Serial Order on Neutral Alumina (Aluminum Oxide, Activated)

By using neutral alumina (aluminum oxide, activated; manufactured by SIGMA-ALDRICH) to prepare tin/alumina impregnating tin to be in an amount of 1 wt %, 0.038 g of tin(II) chloride dihydrate as a tin precursor was put into a beaker to be dissolved in a small amount of hydrochloric acid (0.37 ml) and distilled water (15 ml). When the tin precursor was completely dissolved in the solution prepared as such, 2.0 g of the neutral alumina was added thereto and heated at 70° C. with stirring until the distilled water was completely evaporated. As a result, a solid material remained. Then, the solid material was additionally dried in an oven at 80° C. for 12 hours, and a resultant sample was heat treated in an electric furnace in an air atmosphere at 600° C. for 4 hours. As a result, tin/alumina impregnating tin in an amount of 1 wt % on alumina was obtained.

0.053 g of chloroplatinic acid hexahydrate as a platinum precursor was put into a beaker to be dissolved in distilled water (10 ml) such that a platinum content could be 1 wt % in 2.0 g of the tin/alumina sample obtained as above. When the platinum precursor was completely dissolved in the solution, 2.0 g of the prepared tin/alumina was added to the precursor solution and then stirred at 70° C. until the distilled water was completely evaporated. Then, a remaining solid material was additionally dried in an oven at 80° C. for 12 hours, and a resultant sample was heat treated in an electric furnace in an air atmosphere at 550° C. for 4 hours. As a result, a platinum/tin/alumina catalyst was prepared, and the prepared catalyst was named Pt/Sn/Al₂O₃(N).

### Comparative Preparation Example 2

### Preparation of Platinum/Tin/Alumina (Pt/Sn/Al₂O₃(A)) Catalyst by Impregnating Tin and Platinum in Serial Order on Acidic Alumina (Aluminum Oxide, Activated)

By using acidic alumina (aluminum oxide, activated; manufactured by SIGMA-ALDRICH) to prepare tin/alumina impregnating tin to be in an amount of 1 wt %, 0.038 g of tin(II) chloride dihydrate as a tin precursor was put into a beaker to be dissolved in a small amount of hydrochloric acid (0.37 ml) and distilled water (15 ml). When the tin precursor was completely dissolved in the solution prepared as such, 2.0 g of the acidic alumina was added thereto and heated at 70° C. with stirring until the distilled water was completely evaporated. As a result, a solid material remained. Then, the solid material was additionally dried in an oven at 80° C. for 12 hours, and a resultant sample was heat treated in an electric furnace in an air atmosphere at 600° C. for 4 hours. As a result, tin/alumina impregnating tin in an amount of 1 wt % on alumina was obtained.

0.053 g of chloroplatinic acid hexahydrate as a platinum precursor was put into a beaker to be dissolved in distilled water (10 ml) such that a platinum content could be 1 wt % in 2.0 g of the tin/alumina sample obtained as above. When the platinum precursor was completely dissolved in the solution, 2.0 g of the prepared tin/alumina was added to the precursor solution and then stirred at 70° C. until the distilled water was completely evaporated. Then, a remaining solid material was additionally dried in an oven at 80° C. for 12 hours, and a resultant sample was heat treated in an electric furnace in an air atmosphere at 550° C. for 4 hours. As a result, a platinum/tin/alumina catalyst was prepared, and the prepared catalyst was named Pt/Sn/Al₂O₃(A).

### Comparative Preparation Example 3

### Preparation of Platinum/Tin/Alumina (Pt/Sn/Al₂O₃(WA)) Catalyst by Impregnating Tin and Platinum in Serial Order on Weakly Acidic Alumina (Activated Alumina)

By using weakly acidic alumina (activated alumina; manufactured by SIGMA-ALDRICH) to prepare tin/alumina impregnating tin to be in an amount of 1 wt %, 0.038 g of tin(II) chloride dihydrate as a tin precursor was put into a beaker to be dissolved in a small amount of hydrochloric acid (0.37 ml) and distilled water (15 ml). When the tin precursor was completely dissolved in the solution prepared as such, 2.0 g of the weakly acidic alumina was added thereto and heated at 70° C. with stirring until the distilled water was completely evaporated. As a result, a solid material remained. Then, the solid material was additionally dried in an oven at 80° C. for 12 hours, and a resultant sample was heat treated in an electric furnace in an air atmosphere at 600° C. for 4 hours. As a result, tin/alumina impregnating tin in an amount of 1 wt % on alumina was obtained.

0.053 g of chloroplatinic acid hexahydrate as a platinum precursor was put into a beaker to be dissolved in distilled water (10 ml) such that a platinum content could be 1 wt % in 2.0 g of the tin/alumina sample obtained as above. When the platinum precursor was completely dissolved in the solution, 2.0 g of the prepared tin/alumina was added to the precursor solution and then stirred at 70° C. until the distilled water was completely evaporated. Then, a remaining solid material was additionally dried in an oven at 80° C. for 12 hours, and a resultant sample was heat treated in an electric furnace in an air atmosphere at 550° C. for 4 hours. As a result, a platinum/tin/alumina catalyst was prepared, and the prepared catalyst was named Pt/Sn/Al₂O₃(WA).

### Example 1

### Direct Dehydrogenation of n-Butane Using Continuous-Flow Catalytic Reactor

With the platinum/tin/alumina catalyst prepared by the method of Preparation Example 1, a direct dehydrogenation of n-butane was carried out. Detailed experimental conditions of the reaction were as follows.

A reactant used in the direct dehydrogenation of n-butane of Example 1 was a C4 mixture containing n-butane in an amount of 99.65 wt %, and a composition thereof was as shown in Table 1 below.

TABLE 1
Composition of C4 mixture used as a reactant

| Composition | Molecular formula | wt % |
|---|---|---|
| n-butane | C₄H₁₀ | 99.65 |
| i-butane | C₄H₁₀ | 0.27 |
| 1-butene | C₄H₈ | 0.03 |
| Cis-2-butene | C₄H₈ | 0.05 |

| | |
|---|---|
| Total | 100.00 |

A linear quartz reactor was installed in an electric furnace for the catalytic reaction, and the quartz reactor was filled with the catalyst. In order to activate the catalyst prior to the reaction, a reducing process was carried out. In the reducing process, a temperature of a fixed bed reactor was increased from room temperature to 570° C. and maintained at 570° C. for 3 hours, and a mixed gas for reduction was injected such that a ratio of hydrogen to nitrogen became 1:1, and an amount of the catalyst was set such that an injection rate became 600 cc·hr⁻¹·gcat⁻¹ with respect to the hydrogen. Then, a temperature of the reactor was decreased to 550° C., and the C4 mixture containing n-butane and the nitrogen were allowed to pass through a catalyst layer at 550° C. Direct dehydrogenation of n-butane was carried out as such. In this case, a gas for reaction was injected such that a ratio of the n-butane to the nitrogen became 1:1, and an injection rate was set to be 600 cc·hr⁻¹·gcat⁻¹ with respect to the set amount of the catalyst and the n-butane.

A product contained by-products (methane, ethane, ethylene, propane and propylene) caused by cracking, by-products such as i-butane caused by isomerization, and unreacted n-butane in addition to C4 olefins (1-butene, 2-butene, i-butene, and 1,3-butadiene) as main products of the present reaction. Thus, a gas chromatography was used to separate and analyze the product.

A conversion rate of n-butane, and C4 olefin selectivity and yield in the direct dehydrogenation of n-butane carried out on the platinum/tin/alumina catalyst prepared in Preparation Example 1 were calculated by Equations 1, 2, and 3 as follows.

Conversion rate (%)=(molar amount of reacted n-butane/molar amount of supplied n-butane)×100 [Equation 1]

Selectivity (%)=(molar amount of produced C4 olefins/molar amount of reacted n-butane)×100 [Equation 2]

Yield (%)=(molar amount of produced C4 olefins/molar amount of supplied n-butane)×100 [Equation 3]

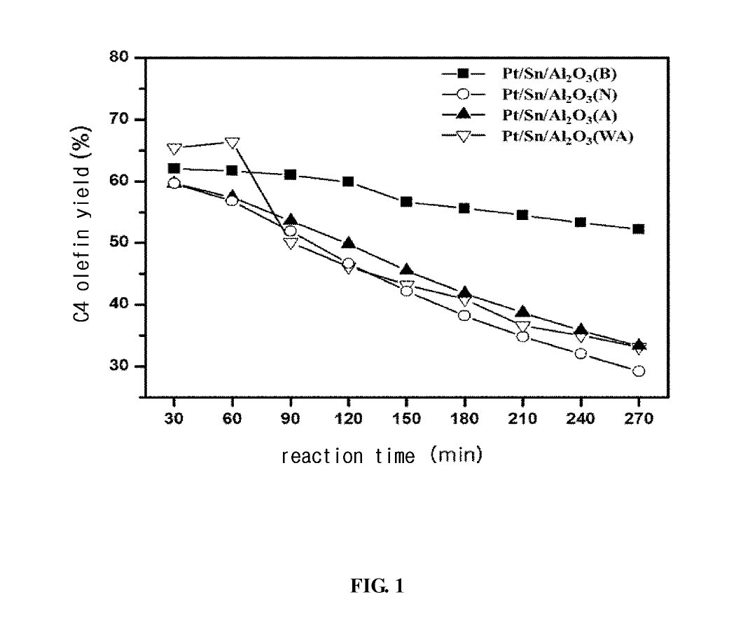

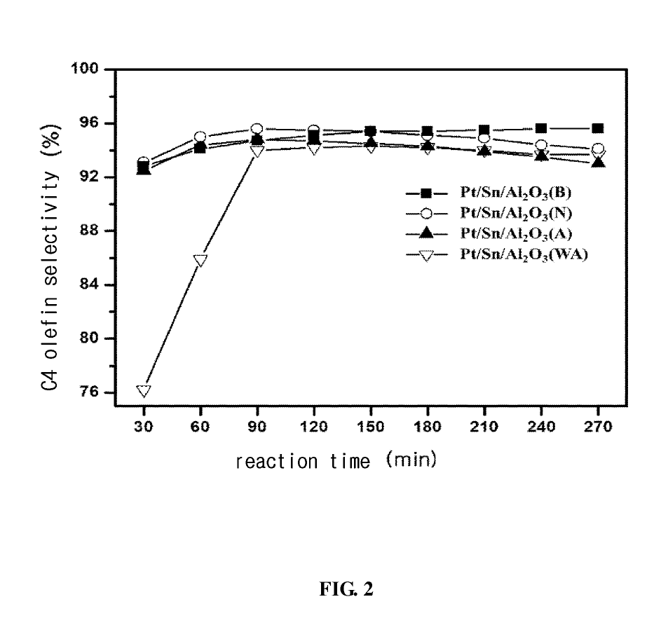

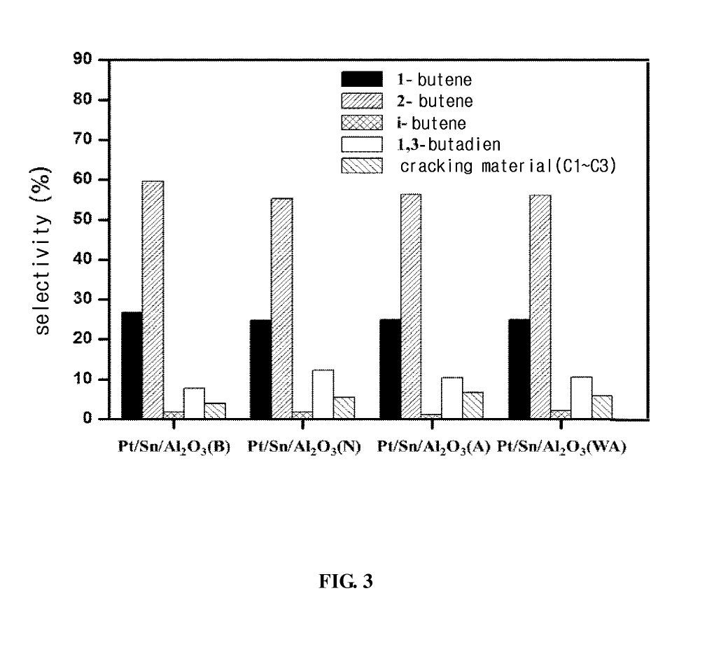

The direct dehydrogenation of n-butane was carried out on the platinum/tin/alumina catalyst prepared in Preparation Example 1 for 270 minutes. Development of reaction activity with time for 270 minutes was as shown in Table 2. Development of change in C4 olefin yield was as shown in FIG. 1 and development of change in C4 olefin selectivity was as shown in FIG. 2. Further, a reaction result at the time of 270 minutes during the reaction was as shown in Table 3 and FIG. 3.

TABLE 2
Development of reaction activity for 270 minutes in direct
dehydrogenation using catalyst Pt/Sn/Al₂O₃(B) prepared
in Preparation Example 1

| Reaction time | N-butane conversion | C4 olefin | C4 |
|---|---|---|---|
| (min) | rate (%) | selectivity (%) | olefin yield (%) |
| 30 | 66.7 | 92.8 | 62.0 |
| 60 | 65.6 | 94.1 | 61.7 |
| 90 | 64.4 | 94.7 | 61.0 |
| 120 | 63.0 | 95.1 | 59.9 |
| 150 | 59.3 | 95.4 | 56.6 |
| 180 | 58.3 | 95.4 | 55.6 |
| 210 | 57.0 | 95.5 | 54.5 |
| 240 | 55.8 | 95.6 | 53.3 |
| 270 | 54.6 | 95.6 | 52.2 |

TABLE 3
Reaction result at 270 minutes during direct dehydrogenation using
catalyst Pt/Sn/Al₂O₃(B) prepared in Preparation Example 1

| | Percentage (%) |
|---|---|
| N-butane conversion rate | 54.6 |

| | | | |
|---|---|---|---|
| Selectivity | 1-butene | 26.7 | 95.6 |
| | 2-butene | 59.6 | |
| | i-butene | 1.7 | |
| | 1,3-butadiene | 7.7 | |

| | |
|---|---|
| i-butane | 0.0 |
| Methane | 0.7 |
| Ethane | 1.1 |
| Ethylene | 0.2 |
| Propane | 0.9 |
| Propylene | 1.0 |

| | |
|---|---|
| C4 olefin yield | 52.2 |

Referring to Table 2, Table 3, and FIGS. 1 to 3, in the case of the direct dehydrogenation of n-butane carried out by using the catalyst Pt/Sn/Al₂O₃(B), deactivation tended to gradually occur as time passed (the conversion rate and yield decreased), whereas the selectivity tended to increase. It is deemed that the deactivation occurred due to coking and deposition as reported in several prior documents. The selectivity of C4 olefins (1-butene, 2-butene, i-butene, and 1,3-butadiene) was as high as 90% or more, and main by-products were cracked materials (methane, ethane, ethylene, propane, and propylene).

### Comparative Example 1

### Reaction Activity in Direct Dehydrogenation of n-Butane Using Platinum/Tin/Alumina Catalysts (Pt/Sn/Al₂O₃(N), Pt/Sn/Al₂O₃(A), and Pt/Sn/Al₂O₃(WA)) Respectively Prepared by Methods of Comparative Preparation Examples 1 to 3

For comparison with the result of reaction activity in the direct dehydrogenation of n-butane using the platinum/tin/alumina catalyst (Pt/Sn/Al₂O₃(B)) prepared by using the basic alumina (aluminum oxide) in Preparation Example 1, direct dehydrogenation of n-butane was carried out in the same manner as Example 1 except that the platinum/tin/alumina catalysts (Pt/Sn/Al₂O₃(N), Pt/Sn/Al₂O₃(A), and Pt/Sn/Al₂O₃(WA)) respectively prepared by using the neutral alumina (aluminum oxide, activated), the acidic alumina (aluminum oxide, activated), and the weakly acidic alumina (activated alumina) by the methods of Comparative Preparation Examples 1 to 3 were used.

A reaction result of Comparative Example 1 was as shown in Tables 4 to 7 and FIGS. 1 to 3. Development of reaction activity with time for 270 minutes in direct dehydrogenation of n-butane using the catalyst Pt/Sn/Al₂O₃(N), the catalyst Pt/Sn/Al₂O₃(A), and the catalyst Pt/Sn/Al₂O₃(WA) was as shown in Table 4, Table 5, and Table 6, respectively. Development of change in C4 olefin yield was as shown in FIG. 1 and development of change in C4 olefin selectivity was as shown in FIG. 2. Further, a reaction result at the time of 270 minutes during the reaction was as shown in Table 7 and FIG. 3.

TABLE 4
Development of reaction activity for 270 minutes in direct
dehydrogenation using catalyst Pt/Sn/Al₂O₃(N) prepared by
method of Comparative Preparation Example 1

| | N-butane conversion | C4 olefin | C4 olefin |
|---|---|---|---|
| Reaction time (min) | rate (%) | selectivity (%) | yield (%) |
| 30 | 64.1 | 93.1 | 59.7 |
| 60 | 59.8 | 95.0 | 56.8 |
| 90 | 54.3 | 95.6 | 51.9 |
| 120 | 48.9 | 95.5 | 46.7 |
| 150 | 44.3 | 95.4 | 42.2 |
| 180 | 40.2 | 95.1 | 38.2 |
| 210 | 36.7 | 94.9 | 34.8 |
| 240 | 33.9 | 94.4 | 32.0 |
| 270 | 31.1 | 94.1 | 29.2 |

TABLE 5
Development of reaction activity for 270 minutes in direct
dehydrogenation using catalyst Pt/Sn/Al₂O₃(A) prepared by method of
Comparative Preparation Example 2

| | N-butane conversion | C4 olefin | C4 olefin |
|---|---|---|---|
| Reaction time (min) | rate (%) | selectivity (%) | yield (%) |
| 30 | 64.4 | 92.5 | 59.6 |
| 60 | 60.9 | 94.4 | 57.4 |
| 90 | 56.6 | 94.8 | 53.6 |
| 120 | 52.6 | 94.7 | 49.8 |
| 150 | 48.1 | 94.5 | 45.5 |
| 180 | 44.3 | 94.3 | 41.8 |
| 210 | 41.2 | 93.9 | 38.7 |
| 240 | 38.3 | 93.5 | 35.8 |
| 270 | 35.8 | 93.0 | 33.3 |

TABLE 6
Development of reaction activity for 270 minutes in direct
dehydrogenation using catalyst Pt/Sn/Al₂O₃(WA) prepared by method of
Comparative Preparation Example 3

| | N-butane conversion | C4 olefin | C4 olefin |
|---|---|---|---|
| Reaction time (min) | rate (%) | selectivity (%) | yield (%) |
| 30 | 85.9 | 76.2 | 65.4 |
| 60 | 77.3 | 85.9 | 66.4 |
| 90 | 53.3 | 94.0 | 50.1 |
| 120 | 48.9 | 94.2 | 46.1 |
| 150 | 45.8 | 94.3 | 43.2 |
| 180 | 43.4 | 94.2 | 40.9 |
| 210 | 39.0 | 94.0 | 36.6 |
| 240 | 37.3 | 93.7 | 35.0 |
| 270 | 35.4 | 93.7 | 33.1 |

TABLE 7
Catalyst activity at 270 minutes during direct dehydrogenation
using catalysts Pt/Sn/Al₂O₃(N), Pt/Sn/Al₂O₃(A), and
Pt/Sn/Al₂O₃(WA) respectively prepared by methods of
Comparative Preparation Examples 1 to 3

| Catalyst | | Pt/Sn/Al₂O₃(N) | | Pt/Sn/Al₂O₃(A) | | Pt/Sn/Al₂O₃(WA) | |
|---|---|---|---|---|---|---|---|
| N-butane conversion | | 31.1 | | 35.8 | | 35.4 | |
| rate (%) | | | | | | | |
| Selec- | 1-butene | 24.8 | 94.1 | 25.0 | 93.0 | 25.0 | 93.7 |
| tivity | 2-butene | 55.3 | | 56.4 | | 56.1 | |
| (%) | i-butene | 1.8 | | 1.1 | | 2.1 | |
| | 1,3- | 12.3 | | 10.4 | | 10.5 | |
| | butadiene | | | | | | |

| | | | |
|---|---|---|---|
| i-butane | 0.0 | 0.0 | 0.0 |
| Methane | 1.0 | 1.2 | 1.0 |
| Ethane | 1.3 | 1.6 | 1.6 |
| Ethylene | 0.6 | 0.5 | 0.5 |
| Propane | 0.4 | 0.7 | 0.6 |
| Propylene | 2.2 | 2.6 | 2.2 |

| | | | |
|---|---|---|---|
| C4 olefin yield (%) | 29.2 | 33.3 | 33.1 |

Referring to Tables 4 to 7, and FIGS. 1 to 3, in the activity experiments of the catalysts respectively prepared in Comparative Preparation Examples 1 to 3, all of the catalysts tended to be deactivated as time passed (the conversion rate and yield decreased), whereas the selectivity tended to increase.

By comparison with the result of Example 1, the catalyst Pt/Sn/Al₂O₃(B) prepared in Preparation Example 1 by impregnating tin and platinum in serial order on basic alumina (aluminum oxide) had higher activity and selectivity than the catalysts (Pt/Sn/Al₂O₃(N), Pt/Sn/Al₂O₃(A), and Pt/Sn/Al₂O₃(WA)) respectively prepared by the methods of Comparative Preparation Examples 1 to 3, and particularly had high C4 olefin selectivity. Further, the results of Example 1 show that a deactivation degree of the catalyst was decreased as time passed.

Accordingly, it was confirmed that the catalyst Pt/Sn/Al₂O₃(B) prepared by impregnating tin and platinum in serial order on basic alumina (aluminum oxide) according to the present invention was most suitable as a catalyst for direct dehydrogenation of n-butane.

### REFERENCES

### Patent Document

Patent Document 1: U.S. Pat. No. 6,433,241 B2 (A. Wu, C. A. Drake) Aug. 13, 2002
Patent Document 2: U.S. Pat. No. 6,187,984 B1 (A. Wu, C. A. Drake) Feb. 13, 2001
Patent Document 3: U.S. Pat. No. 5,344,805 (G. P. Khare, R. A. Porter) May 3, 1993
Patent Document 4: U.S. Pat. No. 4,827,072 (T. Imai, H. Abrevaya, J. C. Bricker, D. Jan) Jul. 20, 1988
Patent Document 5: U.S. Pat. No. 3,960,975 (H. E. Manning) Jun. 3, 1974
Patent Document 6: U.S. Pat. No. 3,960,776 (M. C. Ellis, H. E. Manning) Jul. 1, 1974

### Non-Patent Document

Non-Patent Document 1: A. D. Ballarini, C. G. Ricci, S. R. de Miguel, O. A. Scelza, Catal. Today, volume 133, page 28 (2008)
Non-Patent Document 2: G. Raju, B. M. Reddy, B. Abhishek, Y.-H. Mo, S.-E. Park, Appl. Catal. A, volume 423, page 168 (2012)
Non-Patent Document 3: M. Setnicka, R. Bulanek, L. Capek, P. Cicmanec, J. Mol. Catal. A, volume 344, page 1 (2010)
Non-Patent Document 4: N. Kijima, M. Toba, Y. Yoshimura, Catal. Lett., volume 127, page 63 (2009)
Non-Patent Document 5: J. M. McNamara, S. D. Jackson, D. Lennon, Catal. Today, volume 81, page 583 (2003)
Non-Patent Document 6: S. A. Bocanegra, A. A. Castro, O. A. Scelza, S. R. de Miguel, Appl. Catal. A, volume 333, page 49 (2007)
Non-Patent Document 7: S. A. Bocanegra, S. R. de Miguel, I. Borbath, J. L. Margitfalvi, S. A. Scelza, J. Mol. Catal. A, volume 301, page 52 (2009)
Non-Patent Document 8: S. Veldurthi, C.-H. Shin, O.-S. Joo, K.-D. Jung, Catal. Today, volume 185, page 88 (2012)
Non-Patent Document 9: M. M. Bhasin, J. H. McCain, B. V. Vora, T. Imai, P. R. Pujado, Appl. Catal. A, volume 221, page 397 (2001)
Non-Patent Document 10: S. D. Jackson, S. Rugmini, J. Catal., volume 251, page 59 (2007)
Non-Patent Document 11: Y. Xu, J. Lu, M. Zhong, J. Wang, J. Nat. Gas Chem., volume 18, page 88 (2009)
Non-Patent Document 12: S. Bocanegra, A. Ballarini, P. Zgolicz, 0. Scelza, S. de Miguel, Catal. Today, volume 143, page 334 (2009)
Non-Patent Document 13: A. D. Ballarini, P. Zgolicz, I. M. J. Vilella, S. R. de Miguel, A. A. Castro, O. A. Scelza, Appl. Catal. A, volume 381, page 83 (2010)
Non-Patent Document 14: S. A. Bocanegra, A. A. Castro, A. Guerrero-Ruiz, O. A. Scelza, S. R. de Miguel, Chem. Eng. J., volume 118, page 161 (2006)
Non-Patent Document 15: S. R. de Miguel, S. A. Bocanegra, I. M. Julieta Vilella, A. Guerrero-Ruiz, O. A. Scelza, Catal. Lett., volume 119, page 5 (2007)

1. A preparation method of a platinum/tin/alumina catalyst for direct dehydrogenation of n-butane, comprising the following steps:
(a) preparing a tin precursor solution by dissolving a tin precursor and an acid in a first solvent;
(b) impregnating the tin precursor solution on a basic alumina support;
(c) obtaining tin/alumina in which tin is impregnated on the alumina support by heat drying and heat treating a product obtained from the step (b);
(d) preparing a platinum precursor solution by dissolving a platinum precursor in a second solvent;
(e) impregnating the platinum precursor solution on the tin/alumina prepared in the step (c); and
(f) obtaining a platinum/tin/alumina catalyst by heat drying and heat treating a product obtained from the step (e).

2. The method of claim 1, wherein the tin precursor used in the step (a) includes at least one selected from a group consisting of tin chloride, tin nitride, tin bromide, tin oxide and tin acetate.

3. The method of claim 1, wherein an amount of the tin precursor used in the step (a) is 0.5 to 10 wt % based on a total weight of the final platinum/tin/alumina catalyst.

4. The method of claim 1, wherein the acid used in the step (a) includes at least one selected from the group consisting of a hydrochloric acid, a nitric acid, a sulfuric acid, a hydrofluoric acid and a phosphoric acid.

5. The method of claim 1, wherein each of the first solvent and the second solvent respectively used in the step (a) and the step (d) is water or alcohol.

6. The method of claim 1, wherein in the step (c), the heat drying is carried out at a temperature of 50 to 200° C. and the heat treating is carried out at a temperature of 350 to 1000° C.

7. The method of claim 1, wherein the platinum precursor used in the step (d) includes at least one selected from a group consisting of chloroplatinic acid, platinum oxide, platinum chloride and platinum bromide.

8. The method of claim 1, wherein in the step (f), the heat drying is carried out at a temperature of 50 to 200° C. and the heat treating is carried out at a temperature of 400 to 800° C.

9. A method for producing C4 olefins through direct dehydrogenation of n-butane by using a mixed gas containing n-butane and nitrogen as a reactant on the platinum/tin/alumina catalyst prepared by a method of any one of claims 1 to 8.

10. The method of claim 9, wherein the direct dehydrogenation of n-butane is carried out at a temperature of 300 to 800° C.

11. The method of claim 9, wherein the mixed gas has a volume ratio of the n-butane to the nitrogen is 1:0.2 to 10.

12. The method of claim 9, wherein an injection amount of the mixed gas is set to have a weight hourly space velocity of 10 to 6000 cc·hr⁻¹·gcat⁻¹ with respect to the n-butane.
